(19) United States
(12) Patent Application Publication
(10) Pub. No.: US 2019/0016766 A1
(43) Pub. Date: Jan. 17, 2019

(54) Use of improved modulators of complement function as cancer therapeutics

(71) Applicants: Daniel E. Benjamin, Millstone Township, NJ (US); David C. Fritzinger, Monroe, NJ (US)

(72) Inventors: Daniel E. Benjamin, Millstone Township, NJ (US); David C. Fritzinger, Monroe, NJ (US)

(21) Appl. No.: 16/058,867

(22) Filed: Aug. 8, 2018

Related U.S. Application Data
(63) Continuation-in-part of application No. 15/288,830, filed on Oct. 7, 2016.
(60) Provisional application No. 62/542,611, filed on Aug. 8, 2017.
(60) Provisional application No. 62/238,512, filed on Oct. 7, 2015.

(51) Int. Cl. C07K 14/47; A61P 35/00; C12N 15/62; A61K 9/00

(52) U.S. Cl. CPC C07K 14/47; A61P 35/00; A61K 47/10; A61K 9/0019; C12N 15/62

(57) ABSTRACT

The invention relates generally to derivatives of human C3 protein containing a number of single amino acid changes in the α- and β-chains of C3, designed to increase the affinity of the modified protein to factor B, to lessen the affinity of the modified protein for factor H, and to reduce the immunogenicity of the modified protein as compared to unmodified protein. The invention also presents methods of using these modified proteins for the treatment of certain cancers.

The current application claims a priority to the U.S. Provisional Patent Application Ser. No. 62/542,611, filed Aug. 8, 2017. The current application is Continuation-In-Part of U.S. patent application Ser. No. 15/288,830, filed May 7, 2018, which claims a priority to the U.S. Provisional Patent Application Ser. No. 62/238,512, filed on Oct. 7, 2015.

### FIELD OF INVENTION

The invention relates generally to derivatives of human C3 protein containing a number of single amino acid changes in the α- and β-chains of C3, designed to increase the affinity of the modified protein to factor B, to lessen the affinity of the modified protein for factor H, and to reduce the immunogenicity of the modified protein as compared to the unmodified protein. The invention also presents methods of using these modified proteins for the treatment of certain cancers.

### BACKGROUND OF THE INVENTION

The third component of complement, C3, plays an extremely important role in all three of the activation pathways, the classical, alternative and lectin pathways. In addition, many of the C3 activation products have important functions in immune response and host defense. In all the activation pathways, activated C3, called C3b, is a structural subunit of the C3 convertase. The convertase is formed when C3b binds another complement protein, factor B, which is cleaved by another complement protein, factor D. This yields the activation peptide of factor B, Ba, and Bb, which is bound to C3b to form C3b, Bb. This C3 convertase (C3b,Bb) is then able to activate more C3 molecules in a similar manner. This cleavage also breaks a high-energy thioester linkage, allowing the covalent attachment of C3b to cell surfaces, near the C3 convertase. As more C3 molecules are activated, a tri-molecular complex C3b_{2,}Bb (the C5 convertase), will be formed, which is able to activate C5.

There is a C3 analogue found in cobra venom, called cobra venom factor (CVF) that is a structurally and functionally similar to C3b (Vogel and Muller-Eberhard, 1984). Like C3b, CVF is able to bind factor B, which is cleaved by factor D to yield a C3/C5 convertase, CVF,Bb. Similar to C3b,Bb, CVF,Bb is able to cleave C3 molecules in an identical manner as C3b,Bb. However, the CVF-containing convertase is intrinsically far more stable than the C3b,Bb containing convertase (Janssen et al., 2009; Vogel and Muller-Eberhard, 1982). It is also resistant to regulation by a number of complement regulatory proteins. Finally, unlike C3b,Bb, CVF,Bb acts in the fluid phase rather than on cell surfaces, and is able to activate C5 without binding an additional C3b.

CVF and C3 have also been shown to be quite similar structurally. This similarity is reflected in protein sequence similarity, electron microscopic ultrastructure, and most importantly, three-dimensional structure as determined by x-ray crystallography (Janssen et al, 2009; Rooijakkers et al. 2009, Krishnan et al., 2009). However, there are differences. C3 is a 2-chain molecule with a molecular mass of about 180 kDa, while CVF has a 3-chain structure, with a mass of about 149 kDa, resembling the C3c, one of the breakdown products of C3b (Janssen et al, 2009; O'Keefe et al., 1988).

Because the complement system is involved in many different diseases including some that are wide spread, there has been a great deal of research in drugs that inhibit complement activation. CVF, and CVF-like proteins are unique in that they are able to deplete complement through exhaustive complement activation. Using a C3-like protein with CVF-like properties and low immunogenicity would be a novel means of stopping complement activation (Fritzinger et al., 2009).

Since complement activation has been shown to be important in tumorigenesis and metastasis (Afshar-Kharghan 2017; Rutkowski et al., 2010; Stover 2010), we present several ways in which the use of modified C3 protein that are able to deplete complement will be useful, both as cancer therapeutics and as proteins that may improve the efficacy of monoclonal antibody therapeutics for cancer.

### SUMMARY OF THE INVENTION

The following listing of embodiments is a non-limiting statement of various aspects of the invention. Other aspects and variations will be evident in light of the entire disclosure.

Some embodiments include the replacement of one or more individual amino acids between amino acids 1490 and 1641 (proC3 numbering) of human C3 (SEQ ID NO:1; FIG. 1), such that the modified C3 protein forms a more stable complex with factor B and its activated form, Bb. Some embodiments include the replacement of one or more individual amino acids between positions 730 and 1350, such that the modified C3 has less affinity for factor H short consensus repeats (SCR), 1-4 and SCRs 19-20 (Wu et al., 2009; Bhattacharjee et al., 2011). Some embodiments contain amino acid substitutions between positions 1 and 1642 such that the resulting protein shows less immunogenicity than native C3. Some embodiments will have the amino acid substitutions designed to increase affinity for Bb while decreasing the affinity for factor H. Some embodiments will have amino acid substitutions designed to increase the affinity of the modified C3 protein for Bb while decreasing the immunogenicity of the protein. Some embodiments will have amino acid changes designed to decrease affinity for factor H while decreasing the immunogenicity of the modified C3 protein. Some embodiments will have amino acid substitutions designed to increase the affinity of the modified C3 protein for Bb, decrease the affinity for factor H and decrease the immunogenicity of the resulting protein. In some embodiments, the modified protein will cleave C3 but not C5.

In some embodiments, the modified C3 protein can be expressed as a single chain protein. In some embodiments, the modified C3 protein can be cleaved into at least two chains in a form that resembles C3. In further embodiments, the modified C3 protein can be cleaved to release a C3a portion therefrom. In some embodiments, the amino acids involved in the thioester linkage can be changed, such that the thioester linkage is unable to form. In some embodiments, the entire C3a sequence can be removed, such that the modified protein has the same structure as C3b. In some embodiments, the modified protein can have an additional 1 to 19 amino acids at the N-terminus that are not encoded by C3 or CVF nucleotide. In some embodiments, the modified protein can include a non-C3 signal peptide, such as a Drosophila Bip signal sequence. In some embodiments, the modified C3 protein can have modified affinity for factor B and/or factor D. In some embodiments, the modified protein can show partial or complete resistance to Factor H and/or Factor I. In some embodiments, the modified protein can be essentially non-immunogenic.

Other embodiments can include a method for depleting complement by administering a modified C3 protein to a cancer patient in an amount effective for the depletion of complement. In some embodiments, the administration can be local. In further embodiments, the local administration can be into an organ, subcutaneously, into a cavity, or into a tissue. In other embodiments, the local administration can employ a targeting function capable of concentrating the modified C3 protein in a desired location. In further embodiments, the targeting function can include using an antibody conjugated to the modified C3 protein. In some embodiments, the administration can be a systemic administration, such as intravenous or intraperitoneal.

In some embodiments, the modified protein could also have polyethylene glycol covalently bound to the N-terminus, the C-terminus, or any, some, or all side-chains of lysines. The bound polyethylene glycol would increase the half-life and stability in the blood following parenteral injection, while decreasing immunogenicity. Polyethylene glycol could also be bound to any, some, or all of the other amino acids of the protein. Prior to the polyethylene glycol binding reaction, the active site of the protein could be protected from alteration by pre-incubating with staphylococcal complement inhibitor protein (SCIN).

In some embodiments, complement depletion by the protein(s) described herein will be used to prevent or lessen metastasis of existing tumors. In other embodiments, the proteins will be used to reduce inflammation, thereby reducing tumorigenesis. In other embodiments, the proteins will be used in conjunction with therapeutic monoclonal antibodies to improve their efficacy.

Other embodiments include methods of selecting a modified C3 protein, by characterizing at least one property of the modified C3 protein to form a function profile of the modified protein; and matching the function profile with a disease or condition to be treated. In some embodiments, at least one property can be selected from the group consisting of: convertase activity, convertase formation, convertase stability, susceptibility to Factor H, susceptibility to Factor I, ability to cleave C3, and ability to cleave C5. In some embodiments, the selected C3 protein participates in formation of a convertase adapted for treatment of a chronic condition. In some embodiments, the adaptation can be any of the following: longer plasma half-life, higher stability, greater resistance to Factor H, and greater resistance to Factor I. In some embodiments, the convertase can be adapted for treatment of a reperfusion injury. In other embodiments, the adaptation can be any of, high convertase activity, resistance to Factor H, and resistance to Factor I.

Some embodiments include a nucleic acid sequence encoding a modified C3 protein, and/or a vector including the nucleic acid and/or a host cell containing the vector. In some embodiments, the host cell can be any of the following: a Drosophila S2 cell, an Sf9 cell, a CHO cell, a COS-7 cell, a High Five™ cell, a yeast cell, a BHK cell, and an HEK293 cell.

Some embodiments include a composition that can include the modified human complement C3 protein, or the nucleic acid encoding the modified human complement C3 protein and a pharmaceutically acceptable carrier.

Some embodiments include an expression system expressing the modified C3 protein. In some embodiment, the expression system includes a cell selected from the group consisting of: a Drosophila S2 cell, an Sf9 cell, a CHO cell, a COS-7 cell, a High Five™ cell, a yeast cell, a BHK cell, and an HEK293 cell.

### BRIEF DESCRIPTION OF THE DRAWINGS

FIG. 1A describes the amino acid sequence of human ProC3 (SEQ ID NO:1).

FIG. 1B is a continuation of the amino acid sequence of human ProC3 (SEQ ID NO:1).

FIG. 2A is a continuation of the nucleotide sequence of human ProC3 (SEQ ID NO:2)

FIG. 2B is a continuation of the nucleotide sequence of human ProC3 (SEQ ID NO:2)

FIG. 2C is a continuation of the nucleotide sequence of human ProC3 (SEQ ID NO:2)

FIG. 2D is a continuation of the nucleotide sequence of human ProC3 (SEQ ID NO:2)

FIG. 2E is a continuation of the nucleotide sequence of human ProC3 (SEQ ID NO:2)

### DETAIL DESCRIPTION OF THE INVENTION

Previous studies on human C3/CVF hybrid proteins have shown it is possible to prepare C3 proteins with substitutions of CVF sequences that have many of the properties of CVF, including formation of a stable convertase, reduced factor H affinity, and low immunogenicity (Fritzinger et al., 2009). Combining these studies with the available crystal structures of C3b, CVF, C3b complexed with factor Bb, CVF in a complex with factor B, C3b bound to factor H (SCRs 1 to 4), and C3d bound to factor H (SCRs 19 and 20) (Bhattacharjee et al., 2011) has suggested which individual amino acid substitutions may allow the formation of more stable convertases and which amino acid substitutions may reduce the affinity of C3b for factor H, thereby increasing the stability of modified C3 proteins in vivo. Additionally, computer programs online, such as the Immune Epitope Database from National Institute of Allergy and Infectious Disease (www.iedb.org/home_v3.php) are able to predict which amino acid sequences within a protein increase the immunogenicity of the protein.

### Example 1

### Production of Modified Human C3 Proteins

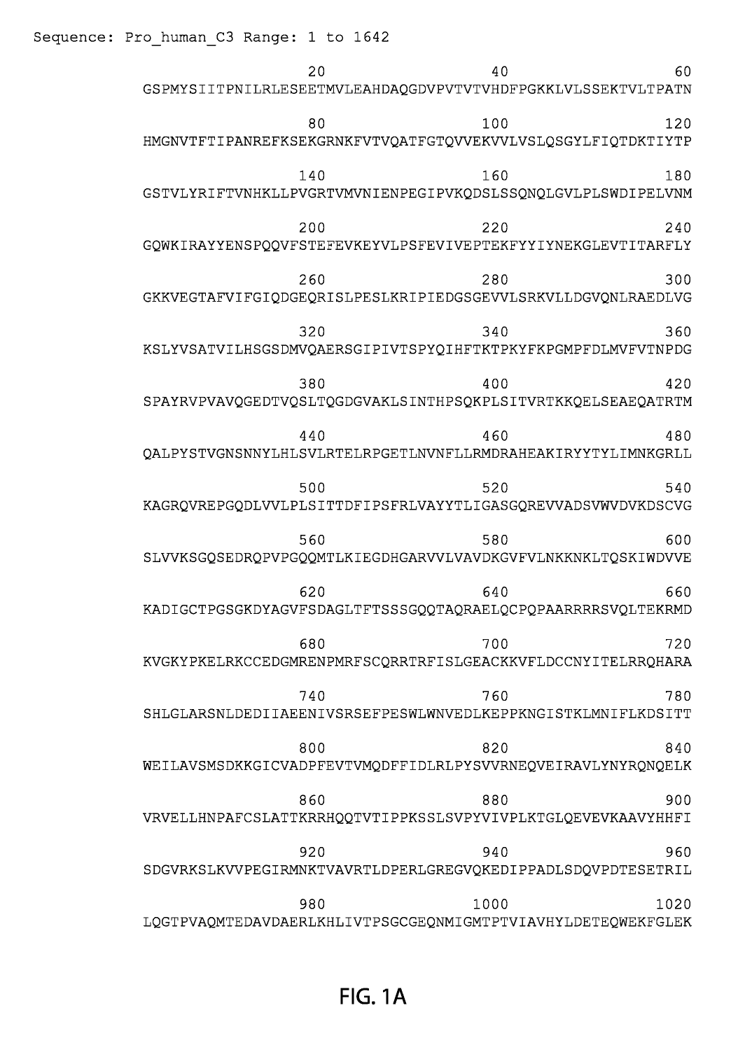

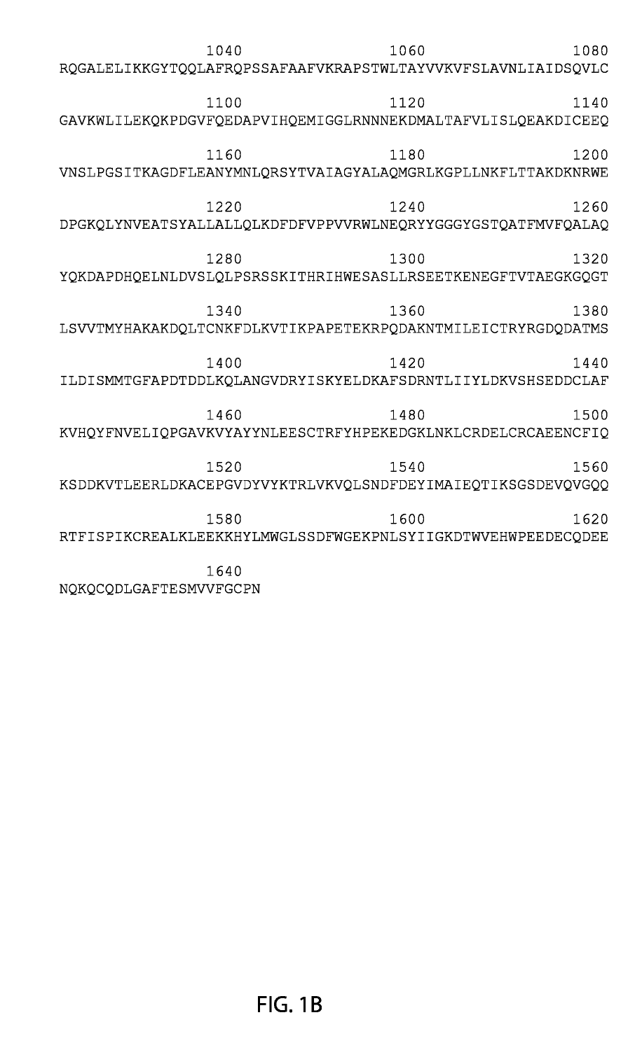

There are numerous amino acid substitutions in human C3 that should produce a modified human C3 that would have a higher affinity for factor B, thus forming a more stable convertase. What follows is a list of some of the mutations in the human C3 protein (SEQ ID NO:1, FIG. 1) or an active fragment thereof: P1517N, Q, H, S, T, β-hydroxy-norvaline; S1550N, D, E, Q, V, I, L, L-Glu-γ-hydrazide; V1637N, T, S, E, Q, D, mono-4-fluoroglutamic Acid; N1642I, E, D, Q, T, S, β-hydroxy-norvaline; G1519I, T, S, V, L, A, N, Q, H, D, E, L-Glu-γ-hydrazide; A1543Q, S, T, V, L, D, E, N, I, L-threo-β-hydroxy-aspartic Acid; I1544G, K, W, V, 3-fluoro-valine, L-t-butyl-glycine, L-threonine, L-allo-threonine; E1545D, N, Q, S, T, 5,5′,5′-trifluoro-leucine, β-t-butyl-alanine; Q1546S, T, M, A, D, E N, mono-4-fluoro-glutamic Acid, 4,4-difluoro-glutamic acid; T1547A, V, L, I, G, W, 3-fluoro-valine, L-t-butyl-glycine; V1555T, S, D, N, Q, E, L-Glu-γ-hydrazide; Q1556L, I, M, V, G, P, thiazolidine-2-carboxylic acid, thiazolidine-4-carboxylic acid, 3,4-dehydro-proline, L- azetidine-2-carboxylic acid; V1557S, T, N, Q, D, E, R, H, K, L-canavanine; Q1559L, R, H, K, N, Q, S-2-aminoethylcysteine, dehydrolysine; E1633L, M, I, Y, W, F, 3-fluoro-L-tyrosine, and 3-nitro-L-tyrosine; V1636T, S, D, E, β-hydroxynorvaline; A1630Q, N, T, S, D, E, or L-Gluγ-hydrazide.

A modified human C3 that would have a lower affinity for factor H, thus increasing the stability of the modified C3 in vivo. What follows is a list of some of the mutations in the human C3 protein (SEQ ID NO:1) or an active fragment thereof: D733G, A, V; I734F, W, Y; E738S, T; N739D, E, L, I, V; H897D, E, T, S, G, A, V, L, I; H898A, G, V, D, E, I, L; K1030T, S, D, E, M, L, I; T1033G, A, V, L, I; V1049T, N, Q, D, E; Q1140W, Y, F, M, S, T, D, E; T1287R, K, H, N, Q; H1291F, W, Y, I, L, S, T, D, E; K1285P, V, A, G, F, D, E; L1298G, A, or V,

A modified human C3 that would decrease immunogenicity. What follows is a list of some of the mutations in the human C3 protein (SEQ ID NO:1) or an active fragment thereof: E176D, S, T, L; V178A, I, L, M, G; Q182N, S, T, I, V; W183F, Y, M, L; K184H, R, Y; Y1173F, M, L, I; A1174G,V; Q1177N, S, T; M1178L, I, V; R1180H, K, T, S, Q, N; K1182H, R, N, Q, T, S; K1194R, K, Q, N; N1197Q, S, T; W1199F, Y, M, L, I; K1204R, H, N, Q; Y1207L, I, M, V; V1232A, G; R1233H, K, N, Q; W1234F, Y, M, L; E1237D; Q1238N, T, S; R1239H, K, N, Q; Y1240F, M, L, I; W781F, Y, M, L V; I783L, M, V, A; L784I, M, V; M788L, I, V, A; K791A, L, N, Q; YSF, W, L, I, M; I7L, V, A; S17T, N, D; R14H, K, N, Q; Y1207F, W, L, M, I; E1210N, S, T; Y1214F, W, I, L, M, V; L12161, M, A, M; Q1221N, T, S; K1223R, H, N, Q; F1252Y, W, L, I, V; M1253L, I, V; F1255Y, W, L, I, V; Q1256N; T; Y1261W, F, M, I, L; K115H, R, Q, N; I117L, V, A; Y118F, W, L, M, I; Y513F, W, L, M, I; Y514F, W L, M, I; L516I, V, A; I517L, V, A; S520T, D, or E.

Stabilization of the protein could also be increased by the attachment of polyethylene glycol on the modified C3 protein.

There are many methods available for site-directed mutagenesis. For high-throughput mutagenesis, one of the best methods is StratageneQuikChange® Site-Directed Mutagenesis Kit. This mutagenesis protocol requires two complementary oligonucleotides, a high-fidelity polymerase, and the restriction enzyme, DpnI. The approach is to hybridize complementary oligonucleotides that contain altered sequence at their center to denatured double-stranded plasmid DNA. A high-fidelity polymerase is used to generate a copy of each strand of the plasmid DNA by priming from the mutagenic primers. This polymerase does not displace the newly synthesized strands and so the extension stops when the primers copy the entire plasmid and return to the 5′ end of the primer. The extension mix is treated with the DNA endonuclease restriction nuclease Dpn1, which requires that the N7 position of adenine be methylated as part its GₘATC recognition sequence. The methylated adenine is only present on the parental plasmid (due to the action of the bacterial DNA methyltransferase, Dam). Thus, DpnI selectively cleaves the parental plasmid DNA, leaving only the mutagenic strands. Once transformed into high efficiency competent bacteria, the annealed mutagenic strand nicks are sealed and the plasmid, now carrying the mutation, is replicated. This method can easily be modified so it will work in 96 well plates, making it possible to adapt it for the high-throughput site-directed mutagenesis needed.

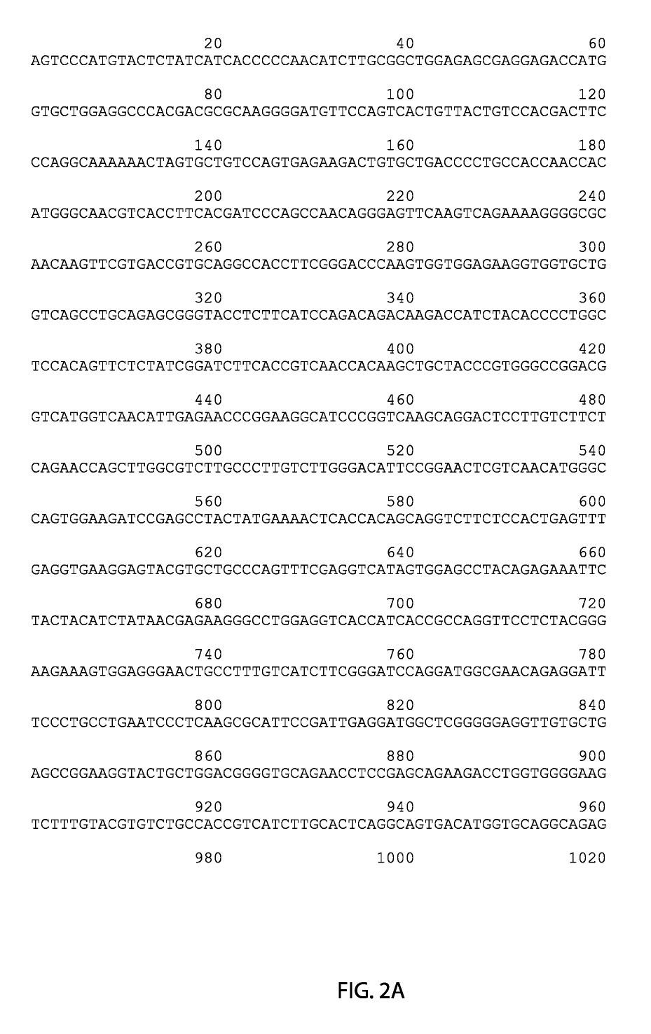

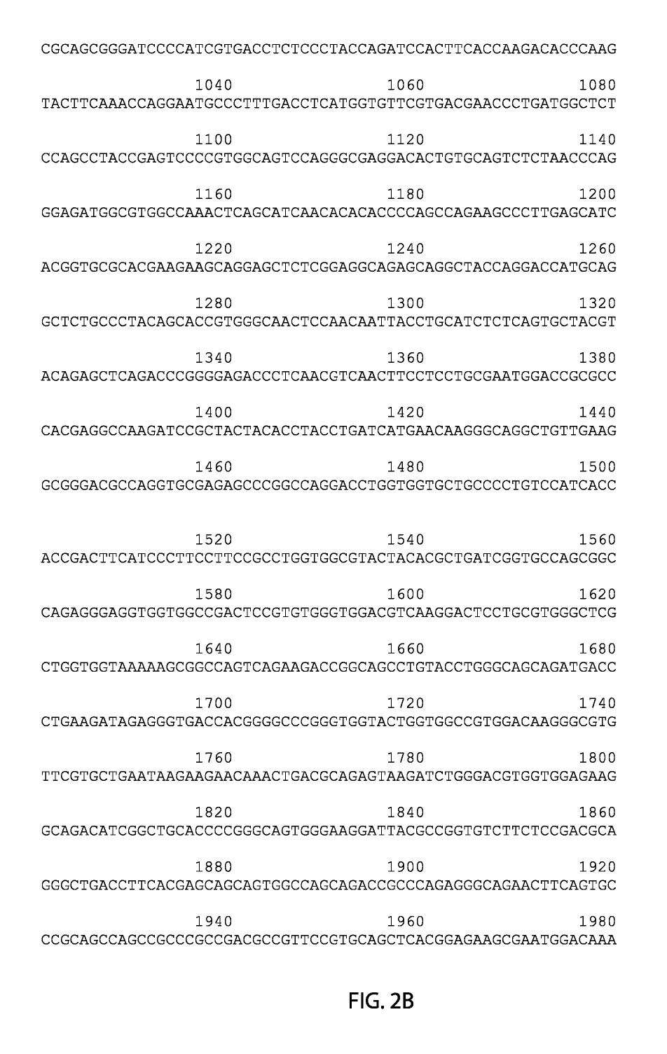

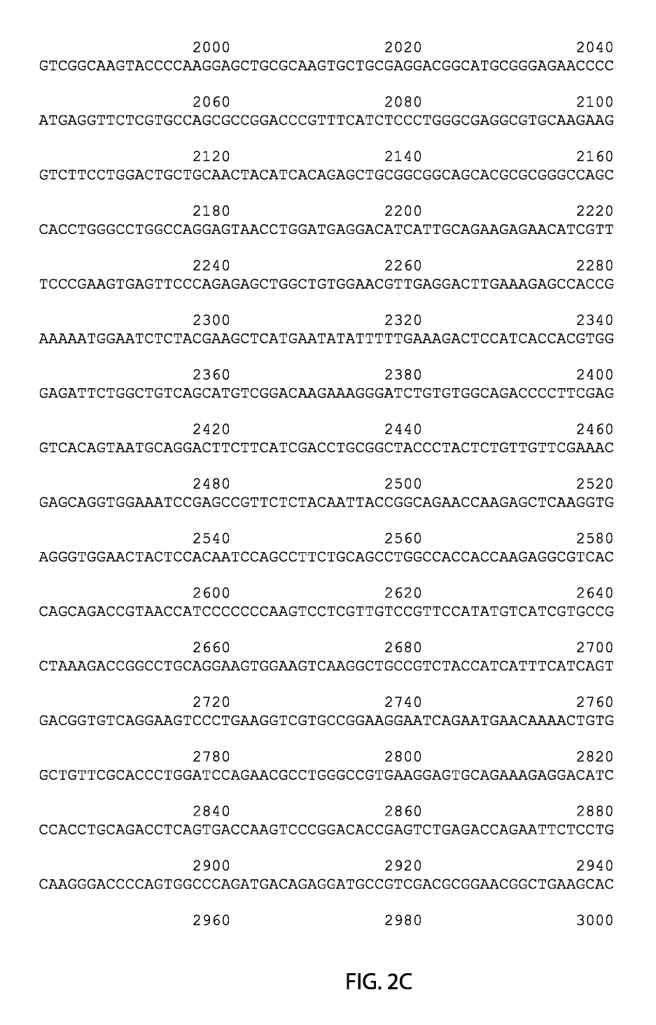

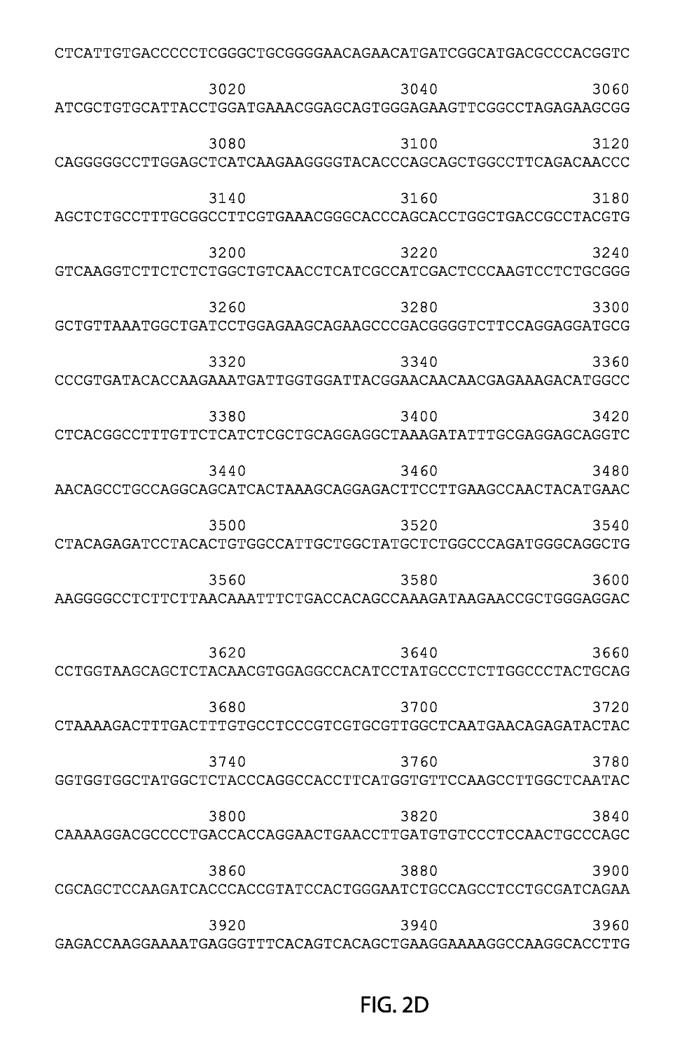

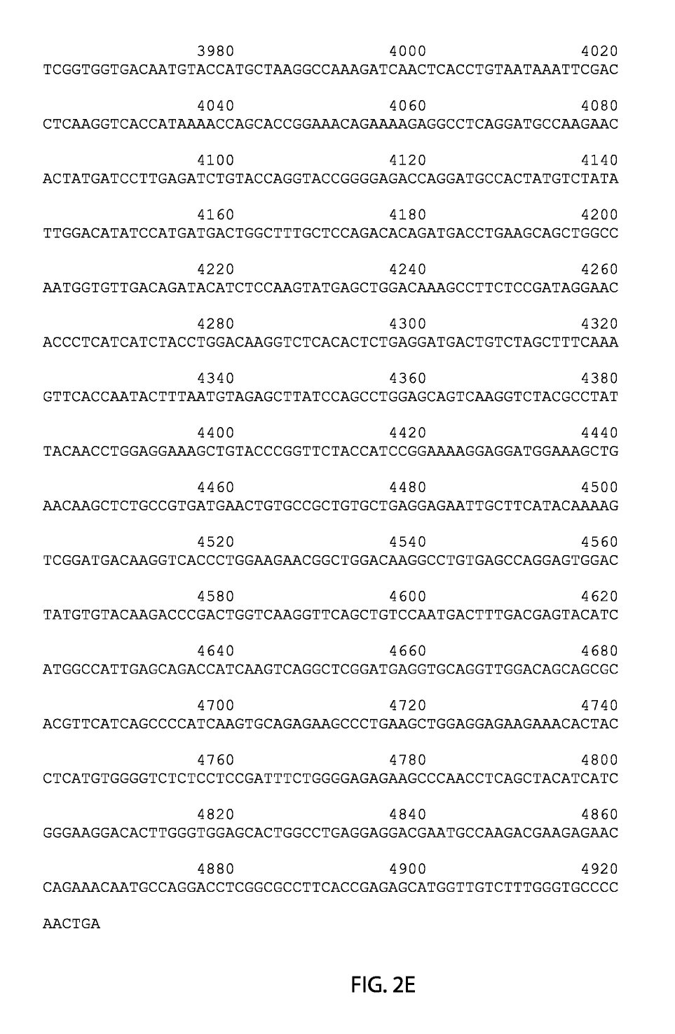

Since the preparation of the proteins described here would require multiple mutations throughout the protein, the fastest method would probably be to use PCR to synthesize the entire protein. This would be done using primers containing the desired mutations, which would then be joined by a second round of PCR amplifications of the coding sequence. The coding sequence for the final construct would then be prepared, either by a final set of PCR amplifications, or by ligating fragments that had been digested with enzymes that have unique restriction sites in the sequence. FIG. 2 describes the nucleotide sequence of human ProC3 (SEQ ID NO:2), which encodes the native human C3 protein (SEQ ID NO:1, FIG. 1).

### Example 2

### Expression of Modified Human C3 Proteins

The modified C3 proteins can be expressed in the quantities needed for evaluation, they will be produced in the Drosophila S2 cell system, using the Drosophila Bip signal sequence for secretion of the proteins, thus eliminating the need for clonal selection. Plasmids containing DNA coding for the modified C3 proteins described above will be cloned into one of the Drosophila S2 expression plasmids (pMT/BiP-V5-HisA, . . . HisB, or . . . HisC). Use of these plasmids allows expression of the proteins with the Drosophila BiP signal sequence, thus ensuring high expression and export in S2 cells. pMT/BiP-V5-HisA, B, or C plasmids containing the coding sequence of a modified C3 protein will be transfected into Drosophila S2 cells using the calcium phosphate method of Chen and Okayama (Chen and Okayama, 1987). S2 cells were transfected with a mixture of expression plasmid and pCoBlast, using a ratio of 19:1 (w:w). Following transfection, cells containing both plasmids were selected using blasticidin (25 μg/ml). For expression, 1-liter cultures of transfected cells were grown in serum-free medium (High Five™ plus L-glutamine), in the absence of blasticidin. When the cells reached a density of 5×10⁶ cells/ml., production of the recombinant proteins was induced by the addition of CuSO₄ to a final concentration of 25 μM. Cultures were allowed to express recombinant proteins for 4-5 days. Hybrid proteins were then purified from the media by a combination of ANX, Sephacryl H-300, and CM-FF chromatography.

### Example 3

### Activity Measurements of Modified Human C3 Proteins

The purified modified human C3 protein hybrids will be subjected to a number of functional analyses as follows.

### Complement Depletion

This assay measures the ability of a protein to deplete complement in human (or other) serum. The assay was done in two steps. In the first step, the protein of interest will be diluted to the desired concentrations in buffer, usually by serial dilution (typically from less than a nanogram/microliter up to approximately 320 ng/microliter or 3.2 μg in the 10 microliters used in the assay). Then, a 10 μL aliquot of the diluted protein will be mixed with undiluted serum. The mixture will be incubated at 37° C. for 3 hours, allowing the protein to exhaustively activate and thus deplete C3 and factor B in the serum. Then, to measure the amount of complement activity left, the serum will be diluted and mixed with antibody-sensitized sheep erythrocytes, which are easily lysed by complement when it is present in serum. This reaction will be allowed to proceed for 30 minutes and will be stopped by diluting the mixture in cold buffer. The cells will be centrifuged and the lysed cells quantified by measuring the hemoglobin released (Fritzinger et al., 2009).

### Factor B Activation Assay

This is an assay to measure the ability of a hybrid protein to activate factor B and form a C3/C5 convertase. The convertase formation will be measured as a function of the cleavage of factor B into Bb and Ba. In the assay, purified modified C3 proteins will be incubated with a three-fold excess of factor B and catalytic amounts of factor D (all highly purified) in the presence of magnesium at 37° C. At various times, aliquots of the reaction will be withdrawn, and the reaction stopped by adding EDTA, which chelates the magnesium. The reaction products will be run on a non-reducing SDS-polyacrylamide gel, which will be stained for proteins with Coomassie Blue. The amount of Factor B converted will be quantified by scanning the gel into a specialized computer program and measuring the amount of protein in the factor B and Bb bands. The results of this assay are dependent on both the rate of factor B activation and the stability of the resulting convertase. Since there is an excess of factor B in the reaction, a very rapid production of Ba and Bb would indicate an unstable convertase (Fritzinger et al., 2009).

### C3 Convertase Activity Assay

This assay measures the activity of C3/C5 convertases containing hybrid proteins to activate human C3, by cleaving off the C3a peptide. To perform this assay, convertases will be formed as described above, and the reaction stopped by the addition of EDTA. The convertase will then be mixed with human C3, and the reaction incubated at 37° C. At the indicated times, aliquots will be removed, and the reaction stopped by mixing with gel loading buffer containing SDS and β-mercaptoethanol. The SDS denatures the proteins, and the β-mercaptoethanol reduces the disulfide bonds between cysteines in the proteins. After electrophoresis under reducing conditions, the gel will be stained with Coomassie Blue dye, and the relative amounts of the C3a-chain and the C3a-chain will be quantified as described above. Care is taken to use the same amount of convertase in each reaction. The results of this assay are dependent both on the activity of the modified C3-containing convertase and its stability, as an active but unstable convertase will rapidly cleave C3, but will stop as the convertase dissociates (Fritzinger et al., 2009).

### Assay for Cleavage of Modified C3 Proteins by Factors H and I

Modified C3 proteins will incubated with purified human Factor H and Factor I at 37° C. for several hours. The reactions are analyzed by subsequent 7% (w/v) SDS polyacrylamide gel electrophoresis under reducing conditions. Factor H binding and factor I activity is determined by the reduction in the strength of the 105 kDa α′-chain band, and appearance of bands with a molecular weight of 37 and 40 kDa (Fritzinger et al., 2009).

### Assays for Immunogenicity

Various methods can be used to analyze immunogenicity, including but not limited to, skin tests, testing the modified C3 protein in transgenic animals which have been genetically engineered to have human immune systems, in vitro methods, including RIA tests using serum generated in such transgenic animals, Radioimmunoprecipitation assays, ELISA assays, Electrochemiluminescence, and Surface Plasmon Resonance. In addition, mouse, rat or guinea pig analogs of some proteins can be constructed, using either mouse, rat or guinea pig C3 sequences. These can be injected into the appropriate animal, and serum is collected and analyzed for the production of antibodies against the modified proteins.

This method measures the stability of the modified C3 protein in plasma in three ways. However, it is to be understood that one or all of the methods can be used as well as any other methods known to one of skill in the art.

The first method measures the stability in serum in vitro. Rabbit serum is isolated and separated from whole blood. Aliquots of different concentrations of the modified C3 proteins that have been biotinylated are added to the serum and allowed to incubate. Aliquots of the serum are removed at various time intervals, and the amount of modified C3 that persists is identified in an ELISA assay using a monoclonal antibody which is specific to human C3. This is one example of animal serum that can be used. The choice of serum will depend on the cross-reactivity of the human C3 antibodies with C3 of that species.

A second method allows the determination of modified C3 in any animal. Modified C3 proteins are biotinylated and injected into an animal. At times, blood is withdrawn from the animal, and serum separated from the blood. The amount of biotinylated protein can be measured by ELISA, using biotin antibodies (or streptavidin), and the activity of the modified protein measured by measuring the amount of C3 remaining in the serum, using the second part of the complement depletion assay to determine remaining complement activity.

### Example 4

### Use of Modified C3 Proteins to Improve the Efficacy of Monoclonal Antibody Treatment of Cancer

There are two ways in which anti-cancer therapeutic monoclonal antibodies kill target cells. In the first, Antibody-Dependent Cellular Cytotoxicity (ADCC), antibody-coated target cells activate natural killer (NK) cells, which phagocytize the antibody coated cells. The second is Complement-dependent Cytotoxicity (CDC), where the antibodies on the target cells activate complement, presumably through the classical pathway, resulting in complement-mediated cell lysis through pathway described above.

The mode of action of Rituximab, B-cell lymphoma-specific monoclonal antibody, has been extensively studied, and in vitro studies indicate that CDC plays a significant role in its mode of action. However, clinical results imply that CDC may not be play as large a role in cytotoxicity as previously thought. A paper by Wang and co-workers (Wang et al., 2009) looked at the ability of Rituximab to lyse cells in the presence (by CDC) or absence (by ADCC) of complement activation. In vitro studies of a mouse model of B-cell lymphoma demonstrated that the presence of active complement lowered the efficacy of a monoclonal antibody similar to Rituximab. Complement was depleted either by heat-treating human serum, or by treating the serum with either CVF or a C3 protein in which the C-terminal 167 amino acid residues are replaced with homologous CVF sequences (called HC3-1496). Then, lymphoma carrying mice were treated with either CVF, or HC3-1496.

Mice treated with both the antibody and CVF, or with the antibody plus HC3-1496 showed significantly longer survival compared to untreated mice, or mice treated with antibody alone, or only with CVF or HC3-1496. These experiments demonstrate that complement depletion could play a significant role in antibody treatment of certain cancers.

### Example 5

### Use of Modified C3 Proteins to Reduce Metastasis by Preventing the Synthesis of Sublytic Amounts of MAC Formation

Complement activation affects tumorigenesis and metastasis in several ways. In one example, cancerous cells can avoid complement dependent cytotoxicity by overexpression of regulators of complement activity (RCAs) on the cell surface. Expression of one of these proteins, CD59, prevents the docking of the MAC on cell surfaces, thus preventing cell lysis. However, localized complement activation still allows the formation of a lower, sublytic density of MACs on the cell surface. It has been shown that, rather than lysing cells, sublytic concentrations of MACs on the cell surface increases cell proliferation, and thus metastasis. Sublytic MAC density also has been shown to promote inflammation by attracting phagocytes, which release of inflammatory mediators. Finally, sublytic MAC density is able to block apoptosis. All of these effects are important for both tumorigenesis and cancer metastasis.

Since sublytic densities of the MAC on cell surfaces can have these cancer-promoting effects, there is the possibility that preventing MAC formation may be effective in blocking both tumorigenesis and tumor metastasis. While several antibodies can block C5 activation, a more effective means of blocking MAC formation may be to block C3 activation, which eventually results in C5 activation. The high concentration of C3 in the blood makes the use of any complement inhibitor difficult, at best. However, complement depletion, using proteins similar to sC3 can inactivate complement by depleting C3 and factor B, thus blocking C5 activation. Since depletion is an enzymatic process, the quantity of therapeutic is much lower than that needed for complement inhibitors, making complement depletion a possible therapeutic for tumorigenesis and metastasis.

### Example 6

### Use of Modified C3 Proteins to Reduce Tumorigenesis by Blocking Localized C3 and C5 Activation, Thus Blocking C3a and C5a Production

One of the products of complement activation is the production of the C3a and C5a anaphylatoxins. C3a is able to promote tumorigenesis in several ways, including activation of Erk1/2 and Akt pathways, leading to mitogenesis. The binding of C3a to its receptor, C3aR can lead to the production of IL-6, resulting in cytokine and growth factor production, and can also induce the production of VEGF, leading to angiogenesis, needed to allow blood supply to fast growing tumor cells. Finally, C3a can cause reorganization of the extracellular matrix and chemotaxis, resulting in metastasis.

The production of C5a can cause all of the effects described above, as well as the production of caspase 3 inhibitors, preventing apoptosis, and the inhibition of CD8⁺ cells and the production of Reactive Oxygen and Nitrogen Species (ROS and RNS), which can damage DNA, making the switch to cancer cells more likely.

As described above, there are numerous ways in which the production of the anaphylatoxins C3a and C5a can induce oncogenesis, tumorigenesis and metastasis (Afshar-Kharghan, 2017 and references therein). Additionally, C3a and C5a, as well as the MAC, can contribute to cell dedifferentiation, an important step in oncogenesis (Stover, 2010 and references therein). Other studies have shown that inhibiting complement pathways can reduce tumorigenesis and metastasis (Stover, 2010). A novel way to prevent complement activation is to deplete one or several complement components through exhaustive activation. This takes place systemically, thus lowering the local concentration of the anaphylatoxins at the site of the tumor. In addition, the half-life of the anaphylatoxins is very short (Peake, et al, 1991, Weisdorf, et al, 1981), meaning there will be little increase in anaphylatoxins concentration at the tumor site.

### Example 7

### Use of Modified C3 Proteins to Reduce Tumor Growth by Reducing C3 Concentrations in the Tumor Environment

C3 can serve as both an inhibitor and a promotor of tumorigenesis and metastasis. For example, C3 can enhance complement dependent cytotoxicity and serve as an inhibitor of tumor growth and contribute to apoptotic clearance (Stover, 2010), while it has also been shown that increased synthesis of both C3 and factor B can promote increased growth of some cancers (Riihila et al., 2017). In addition, C3 can increase tumorigeneses, metastasis and angiogenesis in several forms of cancer, including ovarian non-small cell lung cancer, melanoma and glioblastoma (Afshar-Kharghan, 2017). As described above, it was recently shown that C3 and factor B expression are upregulated in Cutaneous Squamous Cell Carcinoma, and down regulating the expression of either C3 of factor B reduced tumor growth (Riihila et al., 2017). While the authors only looked at the effect of C3 and factor B expression on the growth of these tumors, it is likely that the presence of C3 and factor B in the extracellular matrix could have the same effect. Therefore, complement depletion should also be able to reduce tumor size. Since C3 can have opposite effects on tumorigenesis and metastasis, it would be necessary to examine the role of C3 in different types of cancer.

What is claimed is:

1. A human C3 protein (SEQ ID NO 1) or an active fragment thereof modified to form a stable C3 convertase, with one or more amino acid replacements selected from the group consisting of: P1518N, Q, H, S, T, or β-hydroxy-norvaline; S1550N, D, E, Q, V, I, L, or L-Glu-γ-hydrazide; V1637N, T, S, E, Q, D, or mono-4-fluoroglutamic Acid; N16421, E, D, Q, T, S, or β-hydroxy-norvaline; G15191, T, S, V, L, A, N, Q, H, D, E, or L-Glu-γ-hydrazide; A1543Q, S, T, V, L, D, E, N, I, or L-threo-β-hydroxyl-aspartic acid; I1544G, K, W, V, 3-fluoro-valine, L-t-butyl-glycine, L-threonine, or L-allo-threonine; E1545D, N, Q, S, T, 5,5′,5′-trifluoro-leucine, or β-t-butyl-alanine; Q1546S, T, M, A, D, E, N, mono-4-fluoro-glutamic acid, or 4,4-difluoro-glutamic acid; T1547A, V, L, I, G, W, 3-fluoro-valine, L-t-butyl-glycine; V1555T, S, D, N, Q, E, or L-Glu-γ-hydrazide; Q1556L, I, M, V, G, P, thiazolidine-2-carboxylic acid, thiazolidine-4-carboxylic acid, 3,4-dehydro-proline, or L-azetidine-2-carboxylic acid; V1557S, T, N, Q, D, E, R, H, K, or L-canavanine; Q1559L, R, H, K, N, Q, S-2-aminoethylcysteine, or dehydrolysine; E1633L, M, I, Y, W, F, 3-fluoro-L-tyrosine, or 3-nitro-L-tyrosine; V1636T, S, D, E, or β-hydroxynorvaline; and A1630Q, N, T, S, D, E, or L-Glu-γ-hydrazide, wherein the position of the amino acid residue is based on the human Pro-C3 protein numbering.

2. A human C3 protein (SEQ ID NO:1) or an active fragment thereof modified such that its affinity for complement factor H is lessened, thus increasing its half-life in vivo, wherein the modified C3 protein contains one or more amino acid substitutions selected from the group consisting of: D733G, A, or V; I734F, W, or Y; E738S, or T; N739D, E, L, I, or V; H897D, E, T, S, G, A, V, L, or I; H898A, G, V, D, E, I, or L; K1030T, S, D, E, M, L, or I; T1033G, A, V, L, or I; V1049T, N, Q, D, or E; Q1140W, Y, F, M, S, T, D, or E; T1287R, K, H, N, or Q; H1291F, W, Y, I, L, S, T, D, or E; K1285P, V, A, G, F, D, or E; and L1298G, A, or V, wherein the position of the amino acid residue is based on the human Pro-C3 protein numbering.

3. A human C3 protein (SEQ ID NO:1) or an active fragment thereof modified to decrease immunogenicity, wherein the modified protein contains one or more amino acid substitutions selected from the group consisting of: E176D, S, T, or L; V178A, I, L, M, or G; Q182N, S, T, I, or V; W183F, Y, M, or L; K184H, R, or Y; Y1173F, M, L, or I; A1174G, or V; Q1177N, S, or T; M1178L, I, or V; R1180H, K, T, S, Q, or N; K1182H, R, N, Q, T, or S; K1194R, Q, or N; N1197Q, S, or T; W1199F, Y, M, L, or I; K1204R, H, N, or Q; Y1207L, I, M, or V; V1232A, or G; R1233H, K, N, or Q; W1234F, Y, M, or L; E1237D; Q1238N, T, or S; R1239H, K, N, or Q; Y1240F, M, L, or I; W781F, Y, M, L, or V; I783L, M, V, or A; L784I, M, or V; M788L, I, V, or A; K791A, L, N, or Q; Y5F, W, L, I, or M; I7L, V, or A; S17T, N, or D; R14H, K, N, or Q; E1210N, S, or T; Y1214F, W, I, L, M, or V; L1216I, M, or A; Q1221N, T, or S; K1223R, H, N, or Q; F1252Y, W, L, I, or V; M1253L, I, or V; F1255Y, W, L, I, or V; Q1256N, or T; Y1261W, F, M, I, or L; K115H, R, Q, or N; I117L, V, or A; Y118F, W, L, M, or I; Y513F, W, L, M, or I; Y514F, W L, M, or I; L516I, V, or A; I517L, V, or A; and S520T, D, or E, wherein the position of the amino acid residue is based on the human Pro-C3 protein numbering.

4. The modified C3 protein according to claim 1 with further sequence changes to decrease its affinity for complement factor H, wherein the sequence changes contain one or more amino acid substitutions selected from the group consisting of: D733G, A, or V; I734F, W, or Y; E738S, or T; N739D, E, L, I, or V; H897D, E, T, S, G, A, V, L, or I; H898A, G, V, D, E, I, or L; K1030T, S, D, E, M, L, or I; T1033G, A, V, L, or I; V1049T, N, Q, D, or E; Q1140W, Y, F, M, S, T, D, or E; T1287R, K, H, N, or Q; H1291F, W, Y, I, L, S, T, D, or E; K1285P, V, A, G, F, D, or E; and L1298G, A, or V.

5. The modified C3 protein according to claim 1 with further sequence changes to decrease immunogenicity, wherein the sequence changes contain one or more amino acid substitutions selected from the group consisting of: E176D, S, T, or L; V178A, I, L, M, or G; Q182N, S, T ,I , or V; W183F, Y, M, or L; K184H, R, or Y; Y1173F, M, L, or I; A1174G, or V; Q1177N, S, or T; M1178L, I, or V; R1180H, K, T, S, Q, or N; K1182H, R, N, Q, T, or S; K1194R, Q, or N; N1197Q, S, or T; W1199F, Y, M, L, or I; K1204R, H, N, or Q; Y1207L, I, M, or V; V1232A, or G; R1233H, K, N, or Q; W1234F, Y, M, or L; E1237D; Q1238N, T, or S; R1239H, K, N, or Q; Y1240F, M, L, or I; W781F, Y, M, L, or V; I783L, M, V, or A; L784I, M, or V; M788L, I, V, or A; K791A, L, N, or Q; Y5F, W, L, I, or M; I7L, V, or A; S17T, N, or D; R14H, K, N, or Q; E1210N, S, or T; Y1214F, W, I, L, M, or V; L1216I, M, or A; Q1221N, T, or S; K1223R, H, N, or Q; F1252Y, W, L, I, or V; M1253L, I, or V; F1255Y, W, L, I, or V; Q1256N, or T; Y1261W, F, M, I, or L; K115H, R, Q, or N; I117L, V, or A; Y118F, W, L, M, or I; Y513F, W, L, M, or I; Y514F, W L, M, or I; L516I, V, or A; I517L, V, or A; and S520T, D, or E.

6. The modified C3 protein according to claim 2 with further sequence changes to decrease immunogenicity, wherein the sequence changes contain one or more amino acid substitutions selected from the group consisting of: E176D, S, T, or L; V178A, I, L, M, or G; Q182N, S, T, I, or V; W183F, Y, M, or L; K184H, R, or Y; Y1173F, M, L, or I; A1174G, or V; Q1177N, S, or T; M1178L, I, or V; R1180H, K, T, S, Q, or N; K1182H, R, N, Q, T, or S; K1194R, Q, or N; N1197Q, S, or T; W1199F, Y, M, L, or I; K1204R, H, N, or Q; Y1207L, I, M, or V; V1232A, or G; R1233H, K, N, or Q; W1234F, Y, M, or L; E1237D; Q1238N, T, or S; R1239H, K, N, or Q; Y1240F, M, L, or I; W781F, Y, M, L, or V; I783L, M, V, or A; L784I, M, or V; M788L, I, V, or A; K791A, L, N, or Q; Y5F, W, L, I, or M; I7L, V, or A; S17T, N, or D; R14H, K, N, or Q; E1210N, S, or T; Y1214F, W, I, L, M, or V; L1216I, M, or A; Q1221N, T, or S; K1223R, H, N, or Q; F1252Y, W, L, I, or V; M1253L, I, or V; F1255Y, W, L, I, or V; Q1256N, or T; Y1261W, F, M, I, or L; K115H, R, Q, or N; I117L, V, or A; Y118F, W, L, M, or I; Y513F, W, L, M, or I; Y514F, W L, M, or I; L516I, V, or A; I517L, V, or A; and S520T, D, or E.

7. The modified C3 protein according to claim 4 with further sequence changes to decrease immunogenicity, wherein the sequence changes contain one or more amino acid substitutions selected from the group consisting of: E176D, S, T, or L; V178A, I, L, M, or G; Q182N, S, T, I, or V; W183F, Y, M, or L; K184H, R, or Y; Y1173F, M, L, or I; A1174G, or V; Q1177N, S, or T; M1178L, I, or V; R1180H, K, T, S, Q, or N; K1182H, R, N, Q, T, or S; K1194R, Q, or N; N1197Q, S, or T; W1199F, Y, M, L, or I; K1204R, H, N, or Q; Y1207L, I, M, or V; V1232A, or G; R1233H, K, N, or Q; W1234F, Y, M, or L; E1237D; Q1238N, T, or S; R1239H, K, N, or Q; Y1240F, M, L, or I; W781F, Y, M, L, or V; I783L, M, V, or A; L784I, M, or V; M788L, I, V, or A; K791A, L, N, or Q; Y5F, W, L, I, or M; I7L, V, or A; S17T, N, or D; R14H, K, N, or Q; E1210N, S, or T; Y1214F, W, I, L, M, or V; L1216I, M, or A; Q1221N, T, or S; K1223R, H, N, or Q; F1252Y, W, L, I, or V; M1253L, I, or V; F1255Y, W, L, I, or V; Q1256N, or T; Y1261W, F, M, I, or L; K115H, R, Q, or N; I117L, V, or A; Y118F, W, L, M, or I; Y513F, W, L, M, or I; Y514F, W L, M, or I; L516I, V, or A; I517L, V, or A; and S520T, D, or E.

8. The modified C3 protein according to claim 1 with polyethylene glycol covalently bound to the N-terminus, C-terminus, or a lysine residue on the modified protein, wherein the bound polyethylene glycol increases the half-life and stability of the modified protein in the blood when the modified C3 protein is injected parenterally, and reduces the immunogenicity of the modified protein, and wherein prior to the polyethylene glycol binding reaction, the active site of the modified protein is protected from alteration by pre-incubating with staphylococcal complement inhibitor protein (SCIN).

9. The modified C3 protein according to claim 1 with further sequence changes by adding 1 to 19 amino acids of a non-C3 signal peptide to the N-terminus of the modified protein.

10. The modified C3 protein according to claim 9, wherein the non-C3 signal peptide is a Drosophila BiP sequence or a mammalian signal peptide.

11. A composition comprising the modified C3 protein according to claim 1 and a pharmaceutically acceptable carrier.

12. A nucleic acid sequence encoding the modified human C3 protein according to claim 1.

13. A plasmid or viral vector containing the nucleic acid sequence of claim 12, designed for expression of the modified human C3 protein in a host cell.

14. A host cell containing the plasmid or viral vector according to claim 13, wherein the host cell is a mammalian cell, an insect cell or a yeast cell.

15. A method for increasing the efficacy of monoclonal antibody cancer therapeutics, the method comprising administering to a cancer patient an amount of the modified human C3 protein according to claim 1 effective to systemically deplete complement.

16. A method for depleting complement system, the method comprising administering to a cancer patient an amount of the modified human C3 protein according to claim 1 effective to deplete complement, thus reducing chronic inflammation near a tumor and slowing tumor growth.

17. The method according to claim 16, wherein the amount of the modified human C3 protein effective to deplete complement and to prevent angiogenesis in a tumor, thus reducing tumor growth.

18. The method according to claim 16, wherein the amount of the modified human C3 protein effective to deplete complement and to reduce the remodeling of the extracellular matrix near tumors, thus reducing cellular migration and invasion.

19. The method according to claim 16, wherein the amount of the modified human C3 protein effective to deplete complement and to reduce sublytic amounts of MAC formation, thus increasing apoptosis, and limiting cell proliferation and invasion.

20. A method of depleting C3 and factor B, the method comprising administering to a cancer patient an amount of the modified human C3 protein according to claim 1 effective deplete C3 and factor B in the patient, thus to reduce the growth of squamous cell carcinoma, melanoma tumors, and other solid tumors.


SEQUENCE LISTING (2 sequences)

SEQ ID NO: 1 - protein, 1642 aa, Homo sapiens
GSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 2 - DNA, 4926 bp, Homo sapiens
agtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacctccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtcagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgccgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtctttgggtgccccaactga